(19) United States
(12) Patent Application Publication
(10) Pub. No.: US 2016/0339036 A1
(43) Pub. Date: Nov. 24, 2016

(54) Compound with Anti-Cancer Properties

(71) Applicant: Velgene 3 Limited, Szeged (HU)

(72) Inventors: Attila Erdos, Szeged (HU); Erzsebet Raso, Szeged (HU)

(21) Appl. No.: 15/167,031

(22) Filed: May 27, 2016

Related U.S. Application Data
(63) Continuation of application No. PCT/EP2014/076138, filed on Dec. 1, 2014.

(30) Foreign Application Priority Data
Nov. 29, 2013 (GB) 1321127.1

(51) Int. Cl. A61K 31/5377; C07D 295/26

(52) U.S. Cl. CPC A61K 31/5377; C07D 295/26

(57) ABSTRACT

The invention relates to blocking autophagy in the treatment or prevention of various forms of cancer by using the compounds of the invention or their pharmaceutically acceptable salts. Also described are pharmaceutical compositions comprising the compounds of the invention or pharmaceutically acceptable salts thereof. Pharmaceutical compositions may be formulated as, but not limited to, tablets, capsules, solutions and ointments. The invention further relates to suitable pharmaceutical compositions, which contain the compounds of the invention as a combined preparation for simultaneous, separate or sequential use for the treatment and prevention of cancer.

### RELATED APPLICATIONS

This application is a continuation of International Application No. PCT/EP2014/076138, which designated the United States and was filed on Dec. 1, 2014, published in English.

This application claims priority under 35 U.S.C. §119 or 365 to Great Britain, Application No. 1321127.1, filed Nov. 29, 2013.

The entire teachings of the above application(s) are incorporated herein by reference.

The invention relates to blocking autophagy in the treatment or prevention of various forms of cancer by using the compounds of the invention or their pharmaceutically acceptable salts. Also described are pharmaceutical compositions comprising the above compounds of the invention or pharmaceutically acceptable salt thereof. Pharmaceutical compositions may be formulated as, but not limited to, tablets, capsules, solutions and ointments. The invention further relates to suitable pharmaceutical compositions, which contain the compounds of the invention as a combined preparation for simultaneous, separate or sequential use for the treatment and prevention of cancer.

### SUMMARY

It has been recently discovered that autophagy plays a crucial role in the homeostasis of the cell to maintain its normal function. It has been demonstrated that impaired or attenuated autophagic activity can lead to cancer, liver disease, various myopathies and neurodegenerative disorders. The deterioration of the normal level of autophagy might also be responsible for shortening the life span. In parallel, it has also been demonstrated that stimulation of autophagy can lead to longevity.

Although the regulation of autophagy is a complex phenomenon, certain myotubularin proteins play a key role. It has been shown that Myotubularin related proteins MTMR14 and MTMR6 have a central role by blocking autophagy by antagonizing the type III phosphatidylinositol 3-kinase vacuolar protein sorting protein 34 (VPS34). This central mechanism of the pathway is conserved across the various species.

The inventors have demonstrated that effective molecules dose dependently facilitate MTMR14 and/or MTMR6 and subsequently significantly blocks the autophagic activity of the malignant cell. Therefore, it is effective in treating various forms of cancer.

### BACKGROUND OF THE INVENTION

The term ‘Autophagy’ (derived from the Greek “auto” for self and “phagion” for eating) means cellular self digestion. Autophagy is a highly regulated self-degradation process of eukaryotic cells. During autophagy, parts of the cytoplasm are sequestered by a double-membrane structure, thereby forming a vesicle-like structure called an autophagosome. An autophagosome then fuses with a lysosome, and in the resulting structure called an autolysosome the sequestered cargo becomes degraded by lysosomal hydrolases (proteases, nucleases, lipases and glycosylases). The end products of autophagic breakdown can serve as building blocks for synthetic processes or provide energy for the cell under starvation. Thus, autophagy plays an essential role in the renewal of cellular components (macromolecule and organelle turnover) and primarily functions as a cell-protecting mechanism. Autophagic degradation is important in cell growth and proliferation, survival of cells, and in defense against intracellular microorganisms; in humans, diverse age-related pathological conditions such as cancer, neurodegenerative diseases (e.g., Alzheimer, Parkinson and Huntington disease), stroke, sarcopenia, immune deficiency and heart attack involve dysregulated autophagy.

A basic biochemical reaction that mediates the formation of the autophagic (isolation) membrane is catalyzed by a conserved kinase, (type III phosphatidylinositol 3-kinase) PI3K-III. Thus, PI3K-III is a critical component of the autophagic process. This enzyme converts phosphatidyl-inositol-3 phosphate into phosphatidyl-inositol-3,5 bisphosphate. The molecular antagonists of PI3K-III involve certain myotubularin-related (MTMT) phosphatases. These MTMT enzymes can inhibit autophagic degradation. In genetic model systems and cell cultures, inhibition of mtm genes leads to a potent autophagy activation. Loss-of-function mutations in mtm genes can significantly extend lifespan, suppress neuronal cell death, and prevent muscle and other tissues from undergoing atrophy. A myotubularin protein (MTMT14) is implicated in fine tuning of autophagy.

We have aimed to develop specific MTMR14 inhibitors with the potential to stimulate the autophagic process.

### The Role of Autophagy in Physiology and Pathology

During autophagy, parts of the cytoplasm are sequestered into a double-membrane bound structure called an autophagosome, and then delivered into the lysosome lumen for enzymatic degradation. The resulting products of autophagic degradation are later utilized in anabolic processes or as cellular energy. Autophagy is basically responsible for the elimination of damaged or worn-out cellular components (dysfunctional and abnormal macromolecules and organelles). Autophagy also plays a key role in the cellular stress response during starvation, in the regulation of cell growth, division and loss, in aging control and in the defense against intracellular pathogens. Defects in autophagy can lead to the development of various types of tumours, premature aging, various neurodegenerative disorders, muscle atrophy (sarcopenia), stroke, heart failure and infections caused by parasitic bacteria or viruses. Understanding the mechanisms and regulation of autophagy is therefore of utmost importance for biomedical, social and economic reasons. The most common fatal diseases of mankind normally develop at advanced ages. While the role of pathological functioning of several proteins (such as oncoproteins, tumour suppressors or aggregation-prone proteins) in the development of these diseases has been revealed over the past few decades, understanding the molecular and cytological bases of these processes remains at the forefront of current biological research. Therefore, it is clear that the pathological mechanisms underlying cancer, neurodegeneration and muscle atrophy—all of which are complex, multifactorial processes—are yet to be discovered. Interestingly, these diseases with apparently diverse origin, molecular basis and clinical picture have something else in common apart from the fact that they predominantly develop at advanced ages, and it is that they are all caused by damaged cellular components. Such types of molecular damage include dysfunctional, oxidized, misfolded, crosslinked or aggregated macromolecules. For example, oxidation of DNA may lead to single- or double-stranded breaks, and during the repair of these breaks the nucleotide sequence can change. The resulting mutations can trigger uncontrolled cell division. Protein aggregation can also lead to various neurodegenerative processes. Alzheimer's disease, for instance, is caused by the accumulation of β-amyloid and tau proteins, while Parkinson's disease is accompanied by the aggregation of α-synuclein in dopaminergic neurons. It is the gradual age-related accumulation of molecular damage, which drives the aging process.

Normal metabolic processes result in a continuous generation and accumulation of cellular damage. Various enzymes and the mitochondrial respiratory chain all produce reactive oxygen species (ROS), such as oxygen anions, superoxide and hydroxyl radicals, peroxides, which can oxidize macromolecules. The removal of ROS is essential for the maintenance of cellular homeostasis. Malfunction and deterioration of cellular repair systems are likely to be responsible for aging as well as for the incidence of most age-related diseases. Due to this remarkable molecular convergence, in the near future one may be able to modify (slow down) the rate at which the cells and tissues age and to delay the incidence of numerous age-related degenerative processes. The removal of damaged cellular components primarily occurs through autophagy. During autophagy parts of the cytoplasm are delivered to lysosomes through a regulated process, in which they are degraded by lysosomal hydrolases. Dysfunctional autophagy has been linked to the development of various geriatric diseases (cancer, neurodegenerative disorders, tissue atrophy, heart failure, stroke and microbial infections). Cytological aspects of autophagy were determined many decades ago.

Despite its medical significance, the genetic and molecular basis (that is the regulation and mechanism) of this process were only discovered very recently. There is a quite straightforward explanation for this discrepancy. Autophagic vacuoles are micron-sized and so autophagy in the past century could only be examined by electron microscopy. This idiosyncrasy has made it impossible to use efficient genetic methods (genetic screens) to identify autophagy-specific genes. It is quite obvious why no one undertook the task of detecting autophagy-deficient mutant organisms using electron microscopy. The breakthrough came with the study of autophagy in single-celled yeast. Yeast contains a single autophagic vacuole (an organelle analogous to the lysosome), which can already be identified by light microscopy. This finding was followed by a series of genetic screens to identify yeast autophagy-related genes (ATG). Identification of metazoan orthologs of yeast autophagy genes have opened the way to the molecular and functional (genetic) analysis of autophagy in higher organisms.

During autophagy, cellular components are translocated into the lysosome through a regulated process. Based on the method of translocation, three main types of autophagy can be distinguished: microautophagy, chaperon-mediated autophagy (CMA) and macroautophagy. As used herein ‘autophagy’ encompasses all types of autophagy.

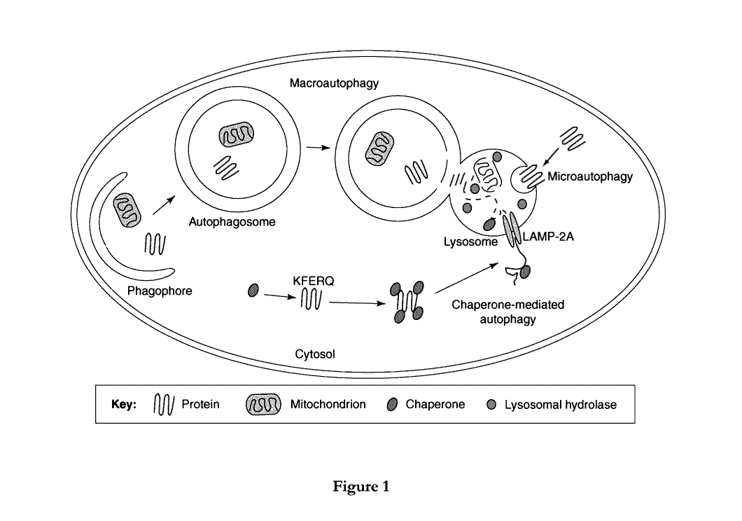

During microautophagy the lysosomal membrane directly engulfs parts of the cytoplasm (invagination). CMA, which does not occur in plant cells, is responsible for the degradation of proteins containing a specific pentapeptide motif, KFERQ. These proteins are marked by molecular chaperones and are transported to the lysosomes though the Lysosome-specific membrane protein type 2a (LAMP-2a) receptor. Interestingly, α-synuclein, whose aggregation results in the development of Parkinson's disease also contains the KFERQ motif. Qualitatively, macroautophagy is the most significant protein and organelle degradation mechanism. During the process of macroautophagy, a double membrane structure is formed inside the cytoplasm, sequestrating cellular components (macromolecules and organelles) from the rest of the cell. When the membrane growing is completed, the resulting structure is called autophagosome (FIG. 1). The mature autophagosome then fuses with a lysosome to form an autolysosome, in which the segregated cellular components are degraded into building blocks.

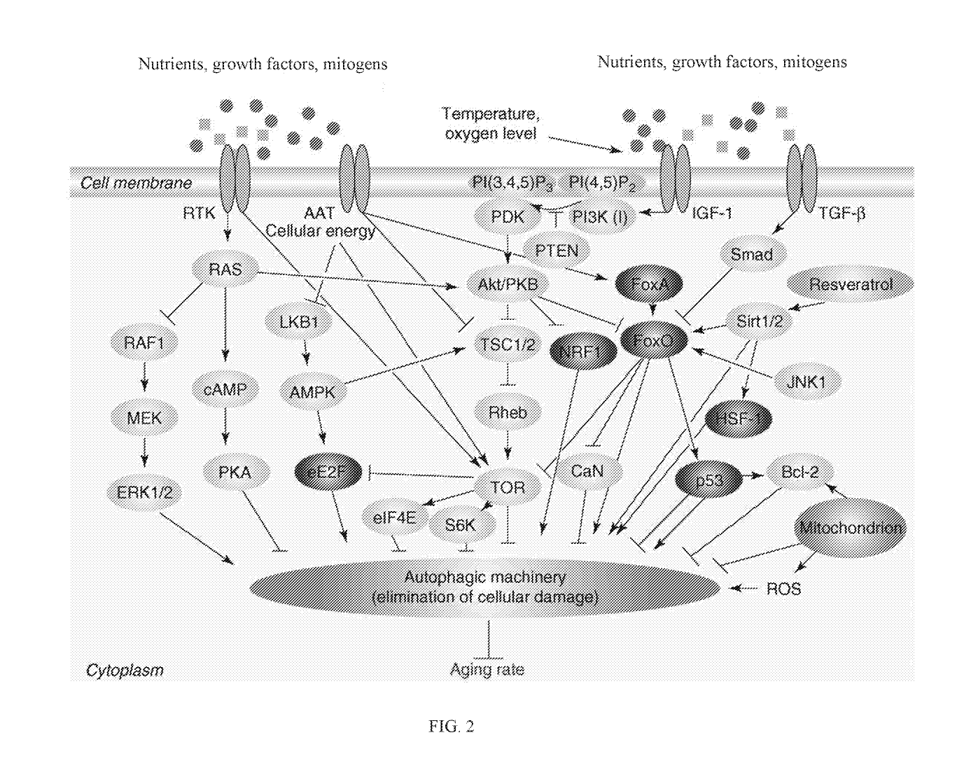

One of the most remarkable features of autophagy is that it is in a tight connection with numerous signal transduction systems, environmental (nutrients, temperature, oxygen) and cellular factors (mitogens, growth factors, ATP levels) (FIG. 2).

Recent results suggest that autophagy acts as a downstream effector process in the regulation of cell growth, proliferation and death. On the other hand, depending on the actual cellular milieu, autophagy is one of the most important means of cell survival. For example, the effect of genetic pathways regulating cell division (such as the Ras, insulin/IGF-1, TGF-β, JNK, G-protein mediated and TOR signal transduction systems) are mediated by the autophagic process. Signal transduction pathways regulating aging (e.g. insulin/IGF-1, TGF-beta, JNK, TOR and Ras/ERK signalling) also converge on the autophagy gene cascade. In addition, biomedically highly important proteins such as p53, FoxO, E2F (a component of the retinoblastoma complex), FoxA, Sirt1 (a sirtuin) regulate the activity of certain autophagy genes directly (i.e. they function as transcription factors of autophagy genes). Therefore, it is evident that autophagy plays a role in the processes of aging, cell division and death.

Autophagy genes are vital in Drosophila and in C. elegans under both normal and starvation-stress induced conditions. In C. elegans, reduced levels of insulin/IGF-1 (insulin-like growth factor 1), TOR signal transduction pathways, mitochondrial respiration or caloric restriction each increase lifespan. The increased lifespan of these animals is autophagy-dependent: inactivation of autophagy genes suppresses the extension of lifespan. Furthermore, it has been demonstrated in insects that the activity (expression) of autophagy genes gradually decreases as the animal ages (as part of the normal aging process) and that overexpression of the autophagy protein Atg8 in the nervous system increases lifespan by 50%. Autophagy genes hence form an “anti-aging” pathway, onto which the effects of the signal transduction systems regulating longevity converge (FIG. 2). Autophagy is, therefore, a central regulatory mechanism of animal aging.

### The Mechanisms of Autophagy

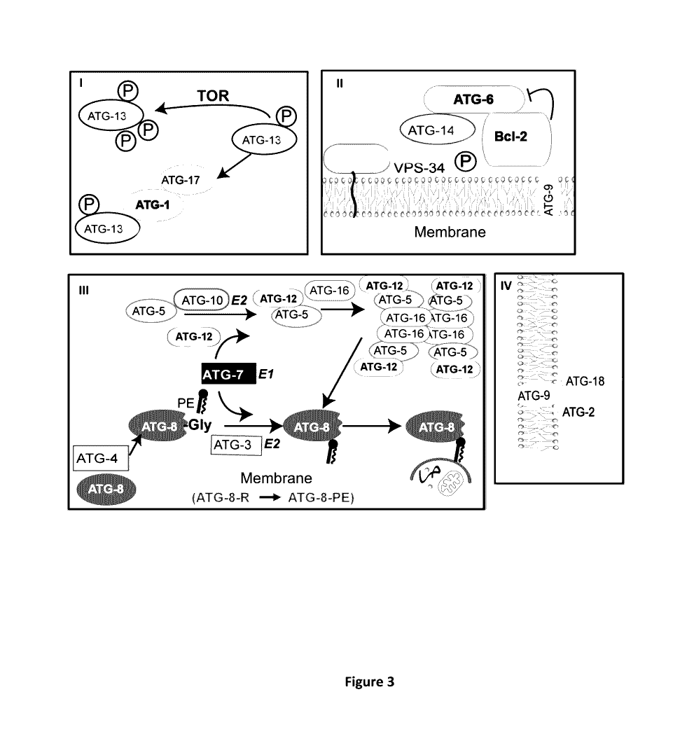

Based on their function, the ATG genes can be classified into four groups: (1) Genes mediating induction (nucleation); (2) Genes that mediate isolation membrane growing; (3) Members of the Atg8 conjugation system; and (4) Genes involved in recyclization (FIG. 3). Induction of autophagy is regulated by an Atg1 kinase complex. This complex contains other proteins, including Atg13 and Atg17. Under normal conditions, Atg13 is phosphorylated by the kinase target of rapamycin (TOR); in this state the complex is not able to initiate autophagy. Under starvation, however, TOR becomes inactivated, resulting in the dephosphorylated state of Atg13. Under these circumstances the Atg1 complex promotes autophagosome formation.

After induction, another kinase complex, whose central component is vacuolar protein sorting-associated protein (VPS34), a type III phosphatidylinositol-3 kinase, mediates the synthesis of the growing isolation membrane. In addition to VPS34, this complex also includes Atg6, Atg14 and Atg15 proteins, and participates in the synthesis of other, non-autophagosomal membranes.

The growing isolation membrane should be identified as an autophagosomal membrane. This can be achieved by covalent binding (conjugation) of Atg8, a ubiquitin-like protein, to the membrane. Initially, Atg8 is a cytosolic, soluble protein (Atg8-I). Upon induction, the last amino acid (a glycine) of Atg8 becomes cleaved off from the protein, leaving a free carboxyl terminus that can bind to a membrane component, phosphatidyl-etanolamine (PE). The PE-bound form of Atg8 is insoluble (Atg8-II). It binds to the forming autophagosomal membranes. In the conjugation process of Atg8, numerous Atg proteins participate, including Atg3, 4, 5, 7, 12 and 16.

After autophagosome formation, its outer membrane fuses with a lysosome, generating thereby a structure called autolysosome, where the cargo (sequestered cytoplasmic materials) is degraded by acidic hydrolases. After autolysosome formation, several components of the autophagoc structure can be regained through recyclization.

### Myotubularin Phosphatases

The catalyst of the initial biochemical process during autophagy is a lipid kinase, PI3K-III, which phosphorylates phosphatidylinositol 3-phosphate (PtdIns3P) to phosphatidylinositol 3,5-bisphosphate (PtdIns3,5P), which is essential for membrane formation. Thus, PI3K-III activity stimulates the formation of autophagosomes. The chemical process catalyzed by PI3K-III is an equilibrated biochemical reaction: myotubularin-related (MTMT) phosphatases dephosphorylate PtdIns3P to PtdIns.

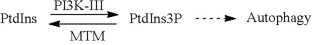

MTMT activity, therefore, results in the suppression of autophagy. This suggests that inhibition of MTMT activity can lead, in theory, to stimulation of autophagy. Indeed, it has been demonstrated that in C. elegans the suppression of certain mtm genes activates autophagy (to salvage the larval mortality of PI3K-III-mutant animals) (Xue et al., 2003).

MTMR proteins form a conserved family of phosphatases. The human genome encodes 13 MTMR proteins (Robinson and Dixon, 2006). These paralogs differ in their structure and only certain types are suitable for efficiently dephosphorylating PtdIns3P. The lack of certain MTMR proteins during ontogeny might lead to the development of mendelian inherited diseases (e.g. myopathy, neuropathy or Charcot-Marie-Tooth syndrome). Out of the 13 human MTMR proteins only MTMR1 (myotubular myophaty), MTMR2 (type 4B1 Charcot-Marie-Tooth syndrome) and MTMR5/13 (infertility in mice) have so far been linked to pathological processes. It is worth mentioning that the lack of MTMR proteins in adulthood (that is, after ontogeny) has not yet been linked to known human disease. This is very important from our point of view and for the concept of the present application: the specific-suppression of MTMR proteins does not result in degenerative disorders.

### DESCRIPTION OF THE INVENTION

In one aspect the present invention relates to the use of autophagy inhibiting compounds of Formula I or their pharmaceutically acceptable salts thereof in blocking autophagy.

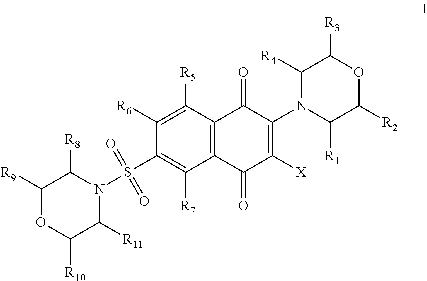

wherein X is halo;
R₁-R₁₁ are each independently selected from H, alkyl, —OH, —NH₂, —NO₂, —COOH, or —CN.

Preferably R₁, R₂, R₃, and R₄ are each independently H or C₁₋₆ alkyl. More preferably R₁, R₂, R₃, and R₄ are each independently H.

Preferably R₈, R₉, R₁₀, and R₁₁ are each independently H or C₁₋₆ alkyl. More preferably R₈, R₉, R₁₀, and R₁₁ are each independently H.

Preferably at least one of R₅, R₆ and R₇ is H. In another embodiment only one of R₅, R₆ and R₇ is not H. In a further embodiment R₅, R₆ and R₇ are all H.

In a preferred embodiment the compound is 1) T0504-7238

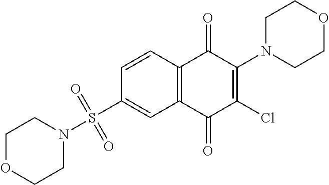 [SMILES: O=C1C(Cl)=C(N2CCOCC2)C(=O)c2ccc(S(=O)(=O)N3CCOCC3)cc21]

The term “halogen atom” or “halo” used herein means a fluorine atom, a chlorine atom, a bromine atom, an iodine atom and the like, preferably a fluorine atom or a chlorine atom, and more preferably a chlorine atom.

The term “alkyl” as used herein, is typically a linear or branched alkyl group or moiety containing from 1 to 20 carbon atoms, such as 11, 12, 13, 14, 15, 16, 17, 18, or 19 carbon atoms. Preferably the alkyl group or moiety contains 1-10 carbon atoms i.e 2, 3, 4, 5, 6, 7, 8, 9, or 10 carbon atoms such as a C₁₋₄ alkyl or a C₁₋₆ alkyl group or moiety, for example methyl, ethyl, n-propyl, i-propyl, n-butyl, i-butyl and t-butyl, n-pentyl, methylbutyl, dimethylpropyl, n-hexyl, 2-methylpentyl, 3-methylpentyl, 2,3-dimethylbutyl, and 2,2-dimethylbutyl.

The invention also relates to a method of blocking autophagy, comprising administering to the subject a therapeutically effective amount of a compound of Formula I or a pharmaceutically acceptable salt thereof. Autophagy is the major catabolic process of eukaryotic cells that degrades and recycles damaged macromolecules and organelles.

“Blocking autophagy” as used herein means decreasing the autophagic activity within a cell or organism as compared to the rate of autophagy in the absence of treatment.

Preferably, the invention relates to an autophagy inhibiting compound of Formula I or a pharmaceutically acceptable salt thereof for use in a method of treating cancer.

The cancer can be selected from, but no limited to melanoma, bone cancer, colon cancer, multiple myeloma, gastric cancer, colorectal cancer, prostate cancer, cervical cancer, lung cancer such as small cell living cancer, non-small cell lung cancer, pancreatic cancer, medulloblastoma, liver cancer, parathyroid cancer, endometrial cancer or breast cancer.

The compounds of the invention may be provided as the free compound or as a suitable salt or hydrate thereof. Salts should be those that are pharmaceutically acceptable and salts and hydrates can be prepared by conventional methods, such as contacting a compound of the invention with an acid or base whose counterpart ion does not interfere with the intended use of the compound. Examples of pharmaceutically acceptable salts include hydrohalogenates, inorganic acid salts, organic carboxylic acid salts, organic sulfonic acid salts, amino acid salt, quaternary ammonium salts, alkaline metal salts, alkaline earth metal salts and the like.

The compounds of the invention can be provided as a pharmaceutical composition. The pharmaceutical composition may additionally comprise a pharmaceutically acceptable excipient for example a pharmaceutically acceptable carrier and/or a pharmaceutically acceptable diluent. Suitable carriers and/or diluents are well known in the art and include pharmaceutical grade starch, mannitol, lactose, magnesium stearate, sodium saccharin, talcum, cellulose, glucose, sucrose (or other sugar), magnesium carbonate, gelatin oil, alcohol, detergents, emulsifiers or water (preferably sterile).

A pharmaceutical composition may be provided in unit dosage form, will generally be provided in a sealed container and may be provided as part of a kit. Such a kit would normally (although not necessarily) include instructions for use. It may include a plurality of said unit dosage forms.

A pharmaceutical composition may be adapted for administration by any appropriate route, for example by the oral (including buccal or sublingual), rectal or topical (including buccal, sublingual or transdermal) route parental, or by inhalation. Such compositions may be prepared by any method known in the art of pharmacy, for example by admixing the active ingredient with a carrier(s) or excipient(s) under sterile conditions.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc. For the preparation of solutions and syrups, excipients which may be used include for example water, polyols and sugars. For the preparation of suspensions oils (e.g. vegetable oils) may be used to provide oil-in-water or water in oil suspensions.

Pharmaceutical compositions adapted for topical administration may be formulated as ointments, creams, suspensions, lotions, powders, solutions, pastes, gels, sprays, aerosols or oils. For infections of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes. Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurised aerosols, nebulizers or insufflators. Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

The pharmaceutical compositions may contain preserving agents, solubilising agents, stabilising agents, wetting agents, emulsifiers, sweeteners, colourants, odourants, salts, buffers, coating agents or antioxidants. They may also contain an adjuvant and/or therapeutically active agents in addition to the substance of the present invention.

Dosages of the substance of the present invention can vary between wide limits, depending upon a variety of factors including the disease or disorder to be treated, the age, weight and condition of the individual to be treated, the route of administration etc. and a physician will ultimately determine appropriate dosages to be used.

Autophagy inhibiting compounds of Formula I for use in the present invention may be administered in combination with one or more other active ingredients known to treat the disease of interest. Compounds of Formula I or a pharmaceutically acceptable salt or hydrate thereof can be adapted for the simultaneous, separate or sequential use with one or more other active ingredients for the treatment and prevention of these diseases.

The invention also relates to methods of blocking autophagy comprising administering an effective amount of a compound of the invention or a pharmaceutically acceptable salt or hydrate thereof, to a subject in need thereof.

The invention will now be described with reference to the following non-limiting examples which refer to the Figures described below.

FIG. 1 shows the main types of autophagy. CMA: chaperon-mediated autophagy, LAMP-2a: Lysosome-Associated Membrane Protein, KFERQ: pentapeptide motif. Dark grey circles stand for mitochondria, curved lines denote proteins, respectively.

FIG. 2 shows the relationship of autophagy with signal transduction pathways and regulatory factors. ROS: Reactive Oxygen Species, PI3K (I): Phosphatidylinositol-3 phosphate kinase I, PI3K (III): Phosphatidylinositol-3 phosphate kinase III. Arrows indicate activation, whereas arrows from bars denote inhibition. Grey circles stand for membrane receptors, yellow circles indicate signalling components, and red circles denote proteins with transcription factor activity, respectively. Basal activity of autophagy prevents cell death, whereas the lack or hyperactivity of autophagy results in cell death (indicated by a combination of arrows and arrows from bars).

FIG. 3 shows the mechanisms of autophagy. Macroautophagy consists of four major steps. A, During induction (nucleation), an Atg1 kinase complex becomes activated which also contains Atg13 and Atg17. Under nutrient deprivation, TOR, a sensor of cellular energy levels, can no longer phosphorylate Atg13. This makes it possible for the complex to activate the formation of the isolation membrane. B, After induction, a “membrane making” complex, the VPS34 kinase complex, mediates the synthesis of the growing isolation membrane. C, A ubiquitin-like protein, Atg8, then binds to the growing membrane. Initially, Atg8 resides in the cytosole in a soluble form (Atg8-I). Upon induction, Atg8 is covalently bind to the membrane, thereby becoming insoluble (Atg8-II). This conjugation process starts with an enzymatic reaction by which the last amino acid (Gly) becomes detached from the carboxyl terminus (C) of Atg8. The activated Atg8 is then covalently linked to a membrane component, PE. The Atg8-PE complex is insoluble, which converts Atg8 as a membrane bound component (Atg8-II). The mature autophagosme fuses with a lysosome, and the resulting structure called autolysosome, serves as a site for the breakdown of cargo.

D, At the end of the process, some components of the autophagic structures can be recyclized.

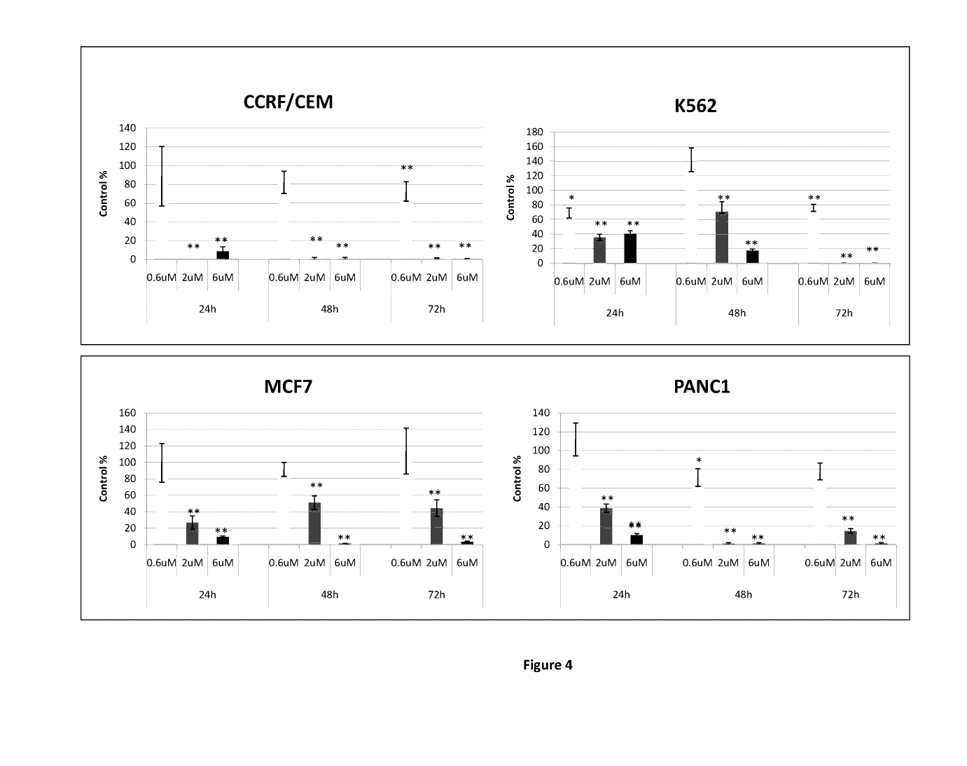

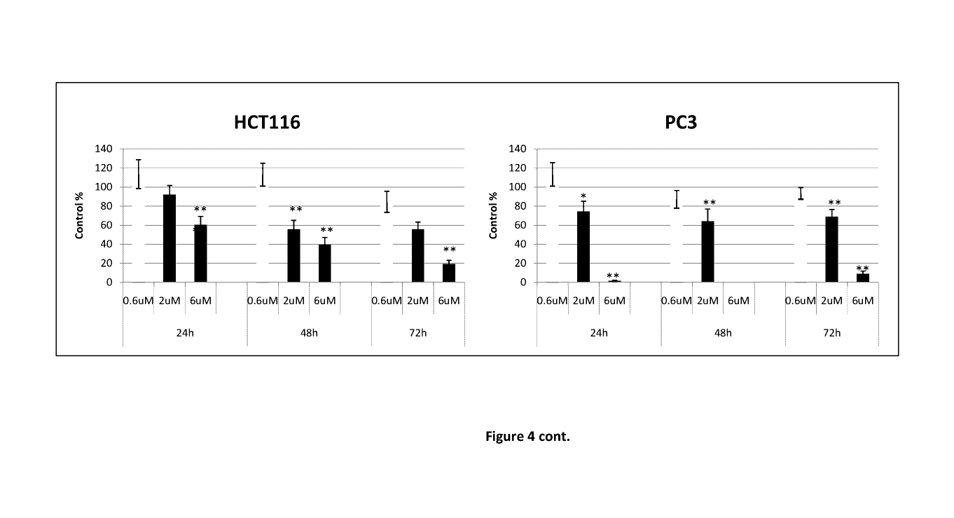

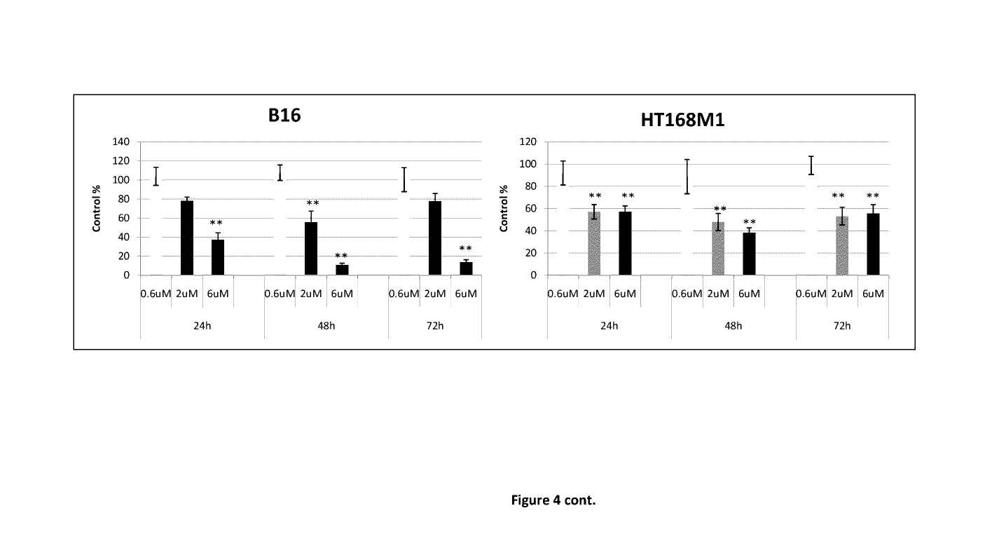

FIG. 4 shows the effect of T0504-7238 on the growth of all tumour cell lines, at a range of doses (0.6 μM-6 μM).

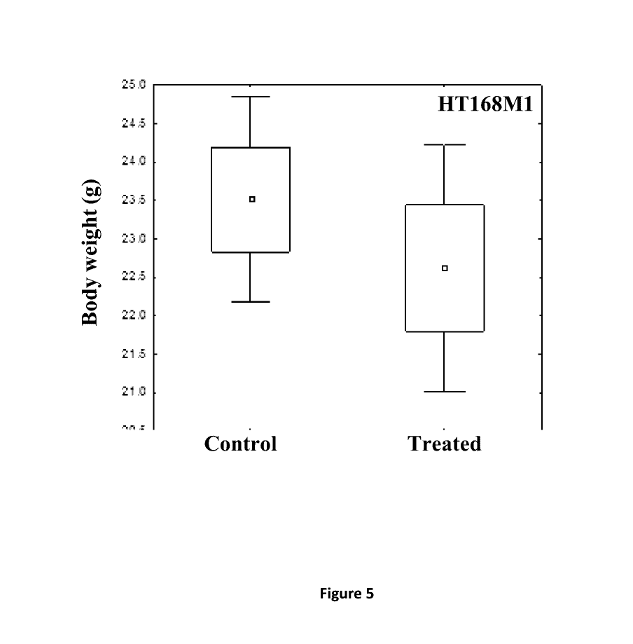

FIG. 5 shows the effect of T0504-7238 on the size and weight of tumours.

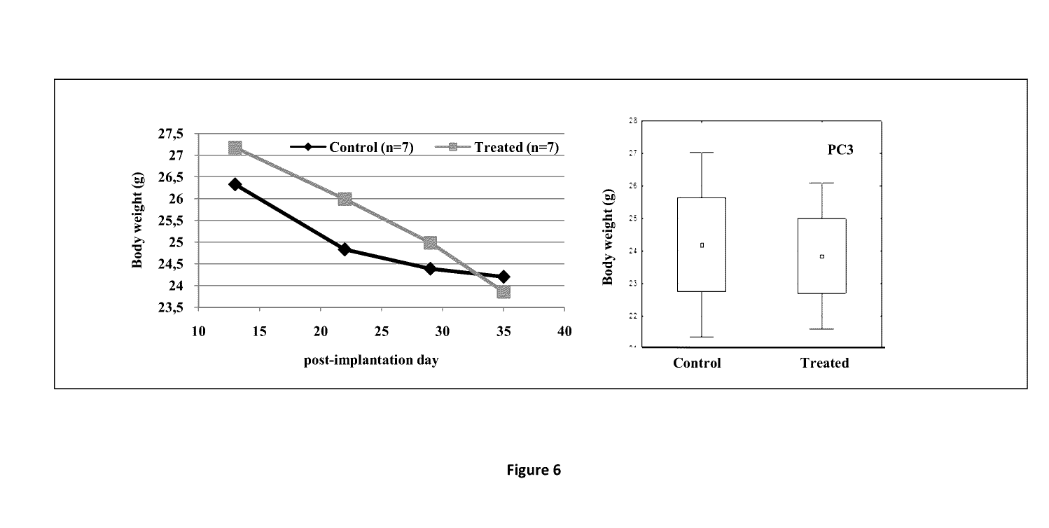

FIG. 6a shows the change in body weight of both the treated and control animals during the course of the experiment. FIG. 6b compares the average body weight.

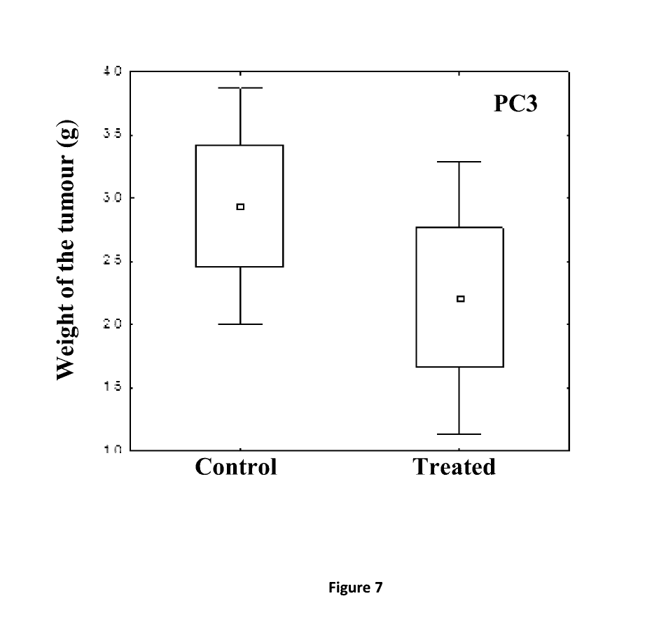

FIG. 7 shows the average weight of the primary tumour in the treated and control animals.

### METHODS

### In Vitro Experiments

The first step was to examine the effect of T0504-7238 on the growth of tumour cells of the cell lines listed below in Table I. Our aim was to select the most therapy-sensitive cell lines as a preparation for the in vivo experiments.

Tumour cells were maintained in RPMI 1640 medium supplemented with 10% FBS (Sigma) and 1% penicillin/streptomycin solution (5,000 units penicillin and 5 mg streptomycin/ml) (Sigma) at 37° C. in a 5% CO₂ atmosphere and used at confluence or at minimum 10⁶ cells/ml density.

TABLE 1
Cell lines

| Cell line | |
|---|---|
| CCRF-CEM | human T cell lymphoblast-like cell line (ALL) |
| K562 | human erythromyeloblastoid leukemia cell line (CML blast |
| | crisis) |
| MCF7 | human breast adenocarcinoma cell line |
| PANC1 | human pancreatic carcinoma, epithelial-like cell line |
| B16 | mouse melanoma cell line |
| HT168M1 | human malignant melanoma cell line |
| (A2058) | |
| HCT116 | human colorectal carcinoma (epithelial) cell line |
| PC3 | human prostate cancer cell line |

### Proliferation Assay

Single cell suspensions of different cell lines (Table 1) were put into 96-well microtiter plates for setting up 18-24 parallel samples per experimental group at 4×10³ cell/well. The appropriate amount of T0504-7238 was suspended in a 3:1 DMSO: Solutol solution, which was then further diluted to the final concentration without the risk of precipitation. All cell lines were tested as non-treated controls, solvent treated controls and samples treated with 0.6, 2 and 6 μM respective dilutions of T0504-7238.

Within each group samples were evaluated 24, 48 and 72 hours after treatment. The colorimetric test was based on the determination of blue/pink formazan generated by mitochondrial reductases of viable cells from MTT (3-(4,5-Dimethylthiazol-2-yl)-2,5-diphenyltetrazolium bromide, Sigma). Briefly, 20 μl of 5 mg/ml MTT solution was added to each well for 4 h at 37° C. After discarding of the culture medium plates were dried at room temperature, formazan crystals dissolved in dimethyl sulfoxide (100 μl/well) and the absorbance (OD) was measured at 570 nm using a microplate reader (Bio-Rad 550).

### Results

The results of our measurements were evaluated using One-Way ANOVA Post Hoc Scheffe Test. We found that, T0504-7238 dose-dependently, significantly decreased the growth of all tumour cell lines, within the dose range examined (0.6 μM-6 μM) as shown in FIG. 4.

### In Vivo Experiments:

Both the environment of the experimental animals and the execution of the actual experiments followed the up to date legislation and ethical regulations of Hungary. The animal experimental unit possesses all the necessary animal welfare certificates. The experimental animals were derived from the The Jackson Laboratory (610 Main Street Bar Harbor, Me. 04609, USA). All personnel involved with the in vivo experiments are qualified to perform the described experiments.

### HT168M1 (Human Melanoma)—Subcutaneous Implantation:

2,5×10⁵ human melanoma (HT168M1) cells, from the in vitro cell line, were subcutaneously (s.c.) implanted into the dorsal region of adult CB17/scid female mice with congenital immunodeficiency under diethyl ether narcosis. The animals were randomised, divided into two groups (11 treated, 11 control) and labelled with individual ear-markings on the 10th post-implantation day. From that day onwards, the treated group was intraperitoneally (i.p) treated with T0504-7238 (2 mg/kg-3:1 DMSO:Solutol solution) once every second day. The control group was also treated with identical frequency to the treatment group i.p. injecting appropriately diluted 3:1 DMSO:Solutol solution. The animals were weighed weekly and individual changes were recorded using the ear-marking system. The tumour diameter was also individually recorded from the earliest palpable stage using a pair of compasses (caliper). Tumour volume was estimated usin the π/6×a×b² formula, where ‘a’ is the longer and ‘b’ is the shorter diameter. The experiment was terminated on the 41st post-implantation day—the animals were sacrificed using Nembutal. The evaluation was based on the weight of the primary tumour.

### Results

The results of our measurements were evaluated using One-Way ANOVA Post Hoc Scheffe Test.

At the time of evaluation, the average weight of the treated animals was found to be lower than that of the controls′, although this difference was not significant. However, there was a significant decrease of body weight in both groups, when compared to the pre-implantation weights (the average body weight of both groups was within standard deviation at the beginning).

The weight of the tumor was found to be significantly lower (p=0.026) in the treated group (see FIG. 5). The detectable tumor in this group was found to be entirely necrotic in 20% of the cases.

PC3 (Human Prostate Tumour)—Implantation into Prostate:

The abdominal wall of CB17/scid male mice was opened at the level of the prostate under Nembutal anaesthesia (75 mg/kg). A single cell suspension (2.15×10⁵ cell/20 μl) of the PC3 human prostate carcinoma cell line was implanted into the prostate using micro syringe (Hamilton). The abdominal was closed with a single layer of surgical thread (3/0 Vicryl), the skin was closed with outwards turning wound edges using surgical clips (Michel Wound Clips, 7.5 mm). After the wound healing completed, the animals were randomised, divided into two groups (9 treated, 9 control) and labelled with individual ear-markings on the 13th post-implantation day. From that day onwards, the treated group was intraperitoneally (i.p) treated with T0504-7238 (2 mg/kg-3:1 DMSO:Solutol solution) once every second day. The control group was also treated with identical frequency to the treatment group i.p. injecting appropriately diluted 3:1 DMSO:Solutol solution. The animals were weighed weekly and individual changes were recorded using the ear-marking system. The experiment was terminated on the 31st post-implantation day—the animals were sacrificed using Nembutal. The evaluation was based on the weight of the primary tumour.

### Results

The results of our measurements were evaluated using One-Way ANOVA Post Hoc Scheffe Test.

### PC3 (Human Prostate Carcinoma)

The body weight of both the treated and control animals significantly decreased during the course of the experiment, however no significant difference was found at the time of evaluation. (See FIG. 6)

The average weight of the primary tumour was lower in the treated group, as shown in FIG. 7.

While this invention has been particularly shown and described with references to preferred embodiments thereof, it will be understood by those skilled in the art that various changes in form and details may be made therein without departing from the scope of the invention encompassed by the appended claims.

1. A compound of Formula I or a pharmaceutically acceptable salt thereof for use in a method of blocking autophagy: 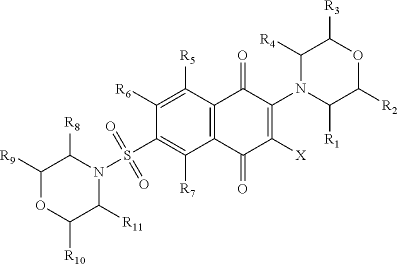
wherein X is halo; and
R₁-R₁₁ are each independently selected from H, alkyl, —OH, —NH₂, —NO₂, —COOH, or —CN.

2. A compound of Formula I or a pharmaceutically acceptable salt thereof for use in a method treating cancer.

3. A compound for use of claim 1, wherein R₁, R₂, R₃, and R₄ are each independently H or C₁₋₆ alkyl.

4. A compound for use of claim 1, wherein R₈, R₉, R₁₀, and R₁₁ are each independently H or C₁₋₆ alkyl.

5. A compound for use of claim 1, wherein at least one of R₅, R₆ and R₇ is H.

6. A compound for use of claim 1, wherein the compound is:

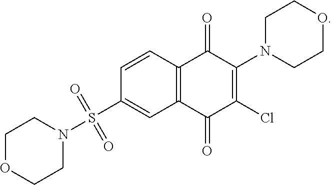 [SMILES: O=C1C(Cl)=C(N2CCOCC2)C(=O)c2ccc(S(=O)(=O)N3CCOCC3)cc21]

7. A compound for use of claim 2, wherein the cancer is selected from melanoma, bone cancer, colon cancer, multiple myeloma, gastric cancer, colorectal cancer, prostate cancer, cervical cancer, lung cancer, pancreatic cancer, medulloblastoma, liver cancer, parathyroid cancer, endometrial cancer or breast cancer.
